(19) United States
(12) Patent Application Publication
(10) Pub. No.: US 2007/0244472 A1
(43) Pub. Date: Oct. 18, 2007

(54) SYSTEM AND METHOD FOR CREATING SUTURE CHANNELS

(76) Inventors: Tobias Kuhn, Heidelberg (DE); Julian Douglas Stevens, London (GB)

Correspondence Address: NYDEGGER & ASSOCIATES, 348 OLIVE STREET, SAN DIEGO, CA, 92103, US

(21) Appl. No.: 11/279,959

(22) Filed: Apr. 17, 2006

(51) Int. Cl. A61B 18/18

(52) U.S. Cl. 606/4

(57) ABSTRACT

A device and method are provided for grafting a donor component onto a recipient member. In the method, a void in the recipient member is defined by a boundary with predetermined dimensions. Similarly, the donor component is specified as having a boundary with predetermined dimensions that are substantially compatible with the void. Thereafter, suture paths are created in the donor component and the recipient member. When the donor component has been positioned in the void of the recipient member, the suture paths are aligned. Then, a suture is passed through the suture path and across the respective boundaries to secure the donor component to the recipient member.

### FIELD OF THE INVENTION

The present invention pertains to ophthalmic surgical transplant procedures that are useful for correcting vision deficiencies. More particularly, the present invention pertains to ophthalmic surgical procedures which incorporate use of a pulsed laser beam for the photoablation and removal of corneal tissue. The present invention is particularly, but not exclusively, useful for removing corneal tissue and for creating a replacement donor graft by photoablating corneal tissue along predetermined boundaries.

### BACKGROUND OF THE INVENTION

Heretofore, a corneal transplant or keratoplasty has been typically preformed using a cylindrical knife called a trephine. During such a transplant, the trephine is used to cut a disc-shaped portion of tissue out of a patient's cornea. The resulting void in the patient's cornea is then filled with a graft of donor tissue having approximately the same dimensions. Thereafter, the graft is secured to the patient's cornea by sutures.

Frequently, several complications arise during such corneal transplants. First, it has been found to be difficult to properly position and hold the eye during the removal of tissue. Typically, the eye has to be grasped at the sclera by forceps. Therefore, the tissue of the eye is stressed during the removal procedure. Second, the trephine itself must apply pressure to the eye in order to make the desired cut. If the eye moves, the cutting maneuver can result in decentration. Further, the deformation of the eye that is caused by the application of pressure can result in non-circular cuts or in badly defined cut edges. Third, the suturing process can cause additional stress to the eye as the needle is passed through corneal tissue. Further, the positioning and quality of the suture is strongly dependent on the skill of the surgeon. As a result of these complications, the healing process and/or the consequent quality of the patient's vision may be impaired.

In light of the above, there is a need for corneal transplant surgical procedures that can be performed without the noted drawbacks attendant to mechanical surgery. Thus, it is an object of the present invention to provide a device and method for performing corneal transplant surgery using photoablation. Another object of the present invention is to provide a device and method for corneal transplant surgery in which the tissue to be replaced and the donor graft have substantially compatible boundaries and dimensions. Another object of the invention is to provide a device and method for performing corneal transplant surgery which utilizes predetermined cut patterns to aid in the healing process. Still another object is to provide a device and method for transplanting corneal tissue in which suture paths are formed in the corneal tissue to reduce the stress on the eye during suturing with needles. Yet another object of the present invention is to provide a method for transplanting corneal tissue which is simple to accomplish and which is relatively cost effective.

### SUMMARY OF THE INVENTION

In accordance with the present invention, a device and method are provided for performing a corneal tissue transplant. Specifically, in the present invention, a section of a recipient member is replaced with a component from a donor member. For the purposes of the present invention, the donor component and the section to be replaced have substantially compatible boundaries and dimensions.

Using available techniques, the precise section to be replaced is defined by a boundary having predetermined dimensions. Typically, the boundary circumscribes all damaged or diseased tissue within the recipient member. With the boundary and the dimensions of the section to be replaced, a substantially compatible volume of donor tissue is also defined. Specifically, the donor component is defined to have substantially the same boundary and dimensions as the section to be replaced, with compensation given for anticipated swelling or contraction of corneal tissue. In this manner, the donor component is able to be fitted precisely within the recipient member. In order to facilitate orientation of the donor component within the recipient, and to facilitate healing, the boundary may define pronged portions that can be symmetrical or asymmetrical.

For purposes of the present invention, once the volume of affected tissue has been predetermined, suture paths are then created in both the recipient member and the donor component. Preferably, the suture paths are photoablated in the recipient member and in the donor component to intersect the respective boundaries of the tissue sections that are to be removed and used for replacement.

After creation of the suture paths, the donor component and the section to be replaced are separated from the donor member and recipient member, respectively. Specifically, the recipient member is photoablated along the boundary of the section to be replaced. As a result of the photoablation, the section to be replaced can be removed from the adjacent portion of recipient member to create a void in the recipient member. Likewise, the donor member is photoablated along the boundary of the component and, thereafter, the component is removed from the donor member.

For the present invention, the component is then positioned in the void in the recipient member. Further, during positioning, the suture paths in the component and in the adjacent portion of the recipient member are aligned. Thereafter, a suture is passed through the suture path and across the respective boundaries to hold the component in position relative to the recipient member.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is schematic drawing of the device of the present invention;

FIG. 2 is an outline of the steps involved in the method of the present invention;

FIGS. 3A-3C are cross sectional views of the corneal tissue of a recipient eye during progressive stages of the method of the present invention;

FIGS. 4A-4B are cross sectional views of the corneal tissue of a donor eye during progressive stages of the method of the present invention;

FIG. 5A is a front view of an exemplary recipient eye shown after a donor corneal tissue has been transplanted in accordance with the present invention;

FIG. 5B is a cross sectional view of the cornea of the eye in FIG. 5A taken along line 5B-5B in FIG. 5A;

FIG. 6A is a front view of an exemplary recipient eye shown after an alternate embodiment of a donor corneal tissue has been transplanted in accordance with the present invention; and

FIG. 6B is a cross sectional view of the cornea of the eye in FIG. 6A taken along line 6B-6B in FIG. 6A.

### DESCRIPTION OF THE PREFERRED EMBODIMENTS

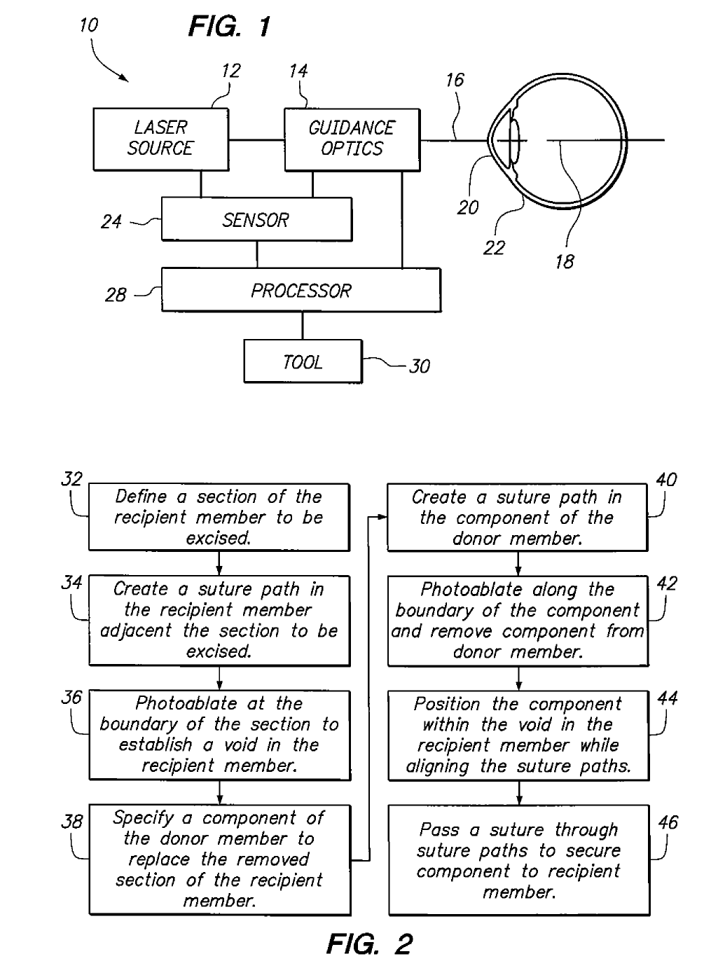

Referring initially to FIG. 1, a device for performing a corneal tissue transplant in accordance with the present invention is shown schematically and is generally designated 10. As shown the device 10 includes a laser source 12 which, preferably, has a photoablation mode in which the laser source 12 generates a continuous train of femtosecond pulses. Specifically, it is necessary that each pulse have an energy level that is above the threshold necessary for the photoablation of corneal tissue. The device 10 also includes guidance optics 14 that are capable of steering and focusing a laser beam 16. As shown, the laser beam 16 is focused along an axis 18 into the corneal tissue 20 of an eye 22. Further, the device 10 may include a sensor 24. The sensor 24 is preferably capable of identifying diseased or damaged portions of corneal tissue 20. As shown, the laser source 12, guidance optics 14, and sensor 24 are interconnected with a processor 28. Preferably, the processor 28 is a dedicated computer that is provided to process data and control the other components of the device 10.

As detailed more fully below, these device components cooperate in combination with each other to photoablate corneal tissue 20 during a corneal transplant procedure. Specifically, the processor 28 is able to use data from the sensor 24 to define a volume of corneal tissue to be bounded by photoablation and to create paths therein by photoablation. Alternatively, this volume can be defined by the surgeon. Thereafter, the processor 28 controls the laser source 12 and guidance optics 14 to perform the desired photoablation. Further, as shown in FIG. 1, the processor 28 is connected to a tool 30 for excising, removing or otherwise manipulating corneal tissue 20 after the photoablation procedure. While shown connected to the processor 28, the tool 30 may be a needle or forceps that are manually operated by a physician to penetrate, excise, or remove corneal tissue 20.

In accordance with the present invention, the device 10 is utilized to transplant transparent material, namely corneal tissue 20, from a donor member to a recipient member. As shown in FIG. 2, the first step in such a procedure is to define a section of the recipient member to be excised (action block 32). Typically, such a section encompasses any damaged or diseased tissue in the recipient member. The section is defined to have a boundary with predetermined dimensions and a resulting volume. For the present invention, the boundary separates the section from the remaining portion of the recipient member.

After the section is defined, a suture path is created in the recipient member by photoablation (action block 34). Specifically, the guidance optics 14 focus the laser beam 16 on corneal tissue 20 to be photoablated. Further, the guidance optics 14 are operated by the processor 28 in accordance with a computer program that is stored in the processor 28. In this manner, the computer program controls photoablation of the corneal tissue 20 along the suture path. Preferably, input from the surgeon can be made into the computer program to perform the specific desired photoablative procedure.

For purposes of the present invention, the suture path intersects the eventual boundary between the section to be excised and the remaining portion of the recipient member. In certain embodiments, the suture path may continue from the boundary to the anterior surface of the recipient member. In alternative embodiments, the suture path may extend from the anterior surface a short distance into the recipient member without reaching the boundary.

After the suture path is created, the corneal tissue along the boundary is photoablated (action block 36). Again, the processor 28 operates the laser source 12 and guidance optics 14 in accordance with a computer program to photoablate the corneal tissue along the boundary. With the boundary photoablated, the section may be photoablated or manually excised from the recipient member to establish a void in the recipient member. At this point, the recipient member is prepared to receive a transplant from the donor member.

Still referring to FIG. 2, preparation of the donor member is now set forth. Specifically, a component of the donor member is specified to replace the removed section of the recipient member (action block 38). For the purposes of the present invention, the component is specified to have a boundary with predetermined dimensions and a resulting volume that are substantially identical to the predetermined dimensions and resulting volume of the removed section and void in the recipient member.

After the component is specified, a suture path is created in the component (action block 40). As above, the suture path is created by photoablation in an operation controlled by the processor 28 and preferably intersects the boundary of the component. In certain embodiments, the suture path may continue from the boundary to the anterior surface of the component. In other embodiments, the suture path may extend a short distance into the component without reaching the anterior surface.

After the suture path in the component is created, the corneal tissue along the boundary is photoablated in an operation controlled by the processor 28 (action block 42). With the boundary photoablated, the component may be removed from the donor member. Thereafter, the component is positioned in the void of the recipient member (action block 44). During positioning, the suture paths in the recipient member and the donor can be aligned to ensure proper orientation of the component. Then a suture is passed through the suture paths and across the boundaries to secure the component to the recipient member (action block 46). Preferably, a needle or other tool is used to pass the suture through the suture paths. Because of the suture paths, the force required to secure the component to the recipient member by suture is significantly reduced. As a result, the recipient member and component undergo less pressure and less risk of complications during recovery.

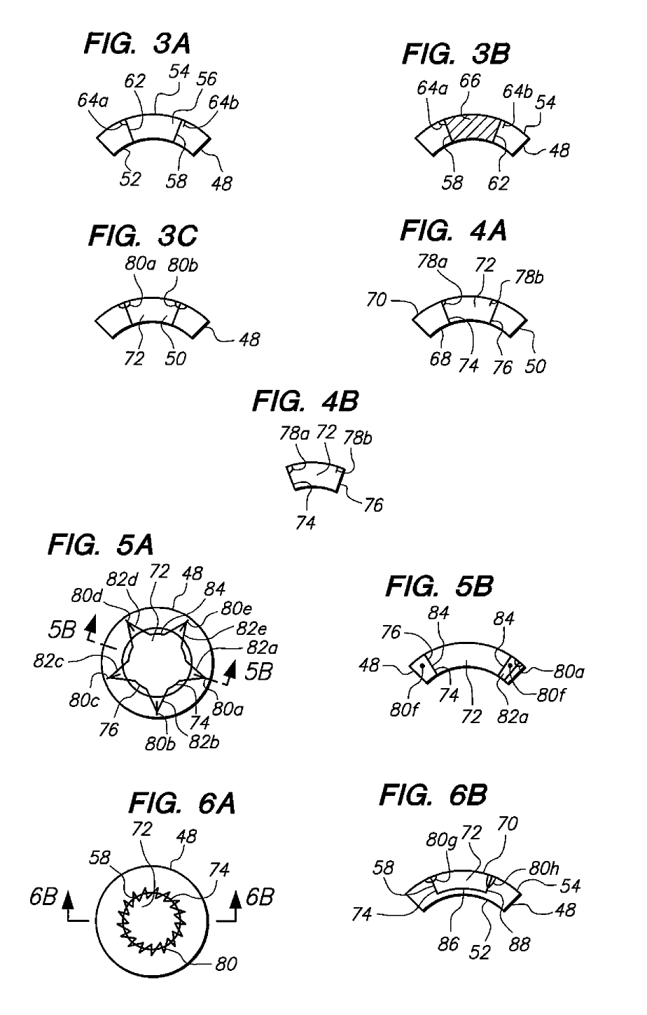

Referring now to FIGS. 3A-3C and 4A-4B, the recipient member 48 and donor member 50 are illustrated at various stages of the operation. Referring first to FIG. 3A, the recipient member 48 is shown after being photoablated. As shown, the recipient member 48 has a posterior surface 52 and an anterior surface 54. Further, the recipient member 48 includes a section 56 to be removed therefrom defined by a boundary 58. As shown, the boundary 58 separates the section 56 from the remaining portion of the recipient member 48. As further shown, the boundary 58 defines predetermined dimensions 62 of the section 56. For the purposes of the present invention, the suture paths 64a-b are formed in the recipient member 48. As shown, the suture path 64a extends from the boundary 58 to the anterior surface 54 of the recipient member 48. On the other hand, the suture path 64b extends into the recipient member 48 from the anterior surface 54 without intersecting the boundary 58. Depending on surgical considerations, either type of suture path 64a and 64b may be used.

Referring now to FIG. 3B, it can be seen that the section 56 has been excised from the recipient member 48. For the present invention, the section 56 may be mechanically excised or entirely photoablated. As a result of the removal of the section 56, a void 66 having the boundary 58 and predetermined dimension 62 is established. It can be further seen that the suture path 64a extends through the recipient member 48 from the boundary 58 to the void 66, while the suture path 64b does not intersect the void 66.

Turning now to FIG. 4A, the preparation of the donor member 50 may be understood. In FIG. 4A, the donor member 50 is shown after being photoablated. As shown, the donor member 50 has a posterior surface 68 and an anterior surface 70. Further, a component 72 to be removed from the donor member 50 includes a boundary 74. As shown, the boundary 74 defines predetermined dimensions 76 of the component 72. For the purposes of the present invention, the predetermined dimensions 76 are substantially compatible with the predetermined dimensions 62 of the section 56 and void 66 of the recipient member 48. As is further shown, suture paths 78a-b are provided in the component 72. Specifically, the suture path 78a extends from the anterior surface 70 to the boundary 74 while the suture 78b extends only into the donor member 50 without intersecting the boundary 74.

Referring now to FIG. 4B, it can be seen that the component 72 has been removed from the donor member 50. It can be further seen that the suture path 78a extends through the component 72 from the anterior surface 70 to the boundary 74, while the suture path 78b does not intersect the boundary 74.

In FIG. 3C, the recipient member 48 is shown after having received the component 72 of the donor member 50. As shown, the component 72 is precisely fitted into the recipient member 48. Further, the suture paths 64a and 78a and the suture paths 64b and 78b are precisely aligned. Also, sutures 80 have been passed through the suture paths 64a and 78a and 64b and 78b to secure the component 72 to the recipient member 48. As illustrated, the suture paths 64a and 78a form a continuous path for the suture 80. On the other hand, the suture paths 64b and 78b are interrupted by corneal tissue 20 at the boundaries 58 and 74. Therefore, during placement of the suture 80 in the suture paths 64b and 78b, the corneal tissue 20 between the suture paths 64b and 78b must be penetrated by a needle or similar tool.

Referring now to FIGS. 5A-5B and 6A-6B, recipient members 48 are shown after receiving components 72 having preferred predetermined dimensions 76. In FIGS. 5A and 5B, the component 72 is shown having a boundary 74 that includes prongs 82a-e that extend radially outward from a substantially circular perimeter 84. While the recipient member 48 and the component 72 are secured to one another by vertical sutures 80a-e, the prongs 82a-e provide for the use of a horizontal suture 80f. Specifically, the horizontal suture 80f travels a substantially circular route radially outside of the perimeter 84. As shown, the horizontal suture 80f passes between the prongs 82a-e and the recipient member 48 to provide further connection therebetween. Moreover, the prongs 82a-e facilitate proper alignment between the recipient member 48 and the component 72.

In FIGS. 6A-6B, the recipient member 48 and the component 72 are shown having boundaries 58 and 74 that are substantially circular. A preferred pattern of sutures 80 extend radially outward and then radially inward in a zigzag fashion. As shown in FIG. 6B, the sutures 80 may include shallow sutures 80g or deep sutures 80h.

In FIGS. 3A-4B and 5A, the results of perforating or penetrating keratoplasty procedures are illustrated. Specifically, in these figures, the sections 56 and components 72 extend from the posterior surfaces 52 and 68 to the anterior surfaces 54 and 70 of the corneal tissue 20. In FIG. 6B, a lamellar graft is illustrated. Specifically, in FIG. 6B, it may be seen that the boundaries 58 and 74 bound a dome-shaped surface 86 that is distanced from both the posterior surface 52 and the anterior surfaces 54 and 70. The boundaries 58 and 74 further include a surface 88 that extends from the posterior surfaces 52 and 68 to the anterior surfaces 54 and 70. In the field of ophthalmic surgery, the surface 86 is referred to as the horizontal surface while the surface 88 is referred to as the vertical surface. For lamellar graft transplants, the horizontal surface 86 is considered to be the component's bed, while the vertical surface 88 is considered to be the component's rim. During the creation of the appropriate component 72 and void 66 in this type of corneal transplant, photoablation is first performed along the horizontal surface 86. Then, the suture paths 64 and 78 are created by photoablation. Thereafter, the vertical surface 88 is photoablated and the transplant is performed.

For the purposes of the present invention, it is preferred that all specific photoablation procedures be performed in an axial direction from the posterior surface 52, 68 to the anterior surface 54, 70 of the subject corneal tissue 20. In other words, photoablation is performed first on the deepest corneal tissue 20 to be photoablated. Thereafter, the focal point of the laser beam 16 is moved toward the anterior surface 54, 70 to photoablate shallower corneal tissue 20. As a result, the gas bubbles are always deeper in the corneal tissue 20 than the focal point of the laser beam 16. In this manner, the laser beam 16 is not forced to pass through the gas bubbles that typically result from the photoablation of corneal tissue 20.

Further, it is envisioned that a wide variety of boundary and suture patterns beyond those depicted in FIGS. 5A and 6A could be employed for the present invention. For example, Zirm, Franceschetti, Sourdille, the Barraquers, Elschnig, the Castroviejos, Katzin, La Roca, Paufique, Carrel, Arruga, and Fritz suture patterns could be employed along with the proper associated boundaries. These specific suture patterns and boundaries are set forth in, and incorporated from, Corneal Grafts, Edited by B. W. Rycroft, Butterworth & Co. (Publishers) Ltd., London, 1955. For purposes of the present invention, these suture patterns and boundaries may be stored in the computer program used by the processor 28. In this manner, the processor 28 may select or suggest an appropriate suture pattern for the surgery to be performed.

While the particular System and Method for Creating Suture Channels as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for transplanting a material from a donor member into a recipient member which comprises the steps of:
defining a boundary surface circumscribing a predetermined volume of material, wherein the boundary surface has specified dimensions;
identifying the boundary surface in the donor member and in the recipient member;
respectively creating a suture path in the donor member and in the recipient member with the suture path intersecting the respective boundary surface at substantially identical points thereon;
photoablating the donor member and the recipient member over their respective boundary surface to create a donor component and a recipient section;
replacing the recipient section with the donor component; and
passing a suture along the suture path to hold the donor component on the recipient member.

2. A method as recited in claim 1 wherein the donor member and the recipient member each have a posterior surface and an anterior surface, and wherein the suture path in the recipient member and the suture path in the component extend from each respective boundary to each respective anterior surface.

3. A method as recited in claim 2 further comprising the step of aligning the suture path in the component with the suture path in the recipient member before passing the suture therethrough.

4. A method as recited in claim 1 wherein the recipient member and the donor member comprise corneal tissue, and further wherein the creating step is performed by photoablating corneal tissue.

5. A method as recited in claim 4 wherein the donor member and the recipient member each have a posterior surface and an anterior surface; and wherein, during the creating step, photoablation is performed in a direction from the posterior surface to the anterior surface.

6. A method as recited in claim 1 wherein the boundary surface defines pronged portions.

7. A method as recited in claim 1 wherein a plurality of suture paths are created in the donor member and in the recipient member with each suture path intersecting the respective boundary surface at substantially identical points thereon.

8. A method for grafting a transparent donor material onto a recipient member comprising the steps of:
defining a void in the recipient member, with said void having a boundary with predetermined dimensions;
creating a suture path in the recipient member;
specifying a component of the donor material, with said component having a boundary with predetermined dimensions substantially compatible with the predetermined dimensions of the void;
positioning the component in the void; and
passing a suture through the component and the recipient member along the suture path to hold the component in position relative to the recipient member.

9. A method as recited in claim 8 further comprising the step of creating a suture path in the component, and wherein, during the passing step, the suture is passed through the suture path in the component and the suture path in the recipient member.

10. A method as recited in claim 9 wherein each suture path intersects a respective boundary, and wherein the method further comprises the step of aligning the suture path in the component with the suture path in the recipient member before passing the suture therethrough.

11. A method as recited in claim 10 wherein the donor material and the recipient member each have a posterior surface and an anterior surface, and wherein each suture path extends from the respective boundary to the respective anterior surface.

12. A method as recited in claim 8 further comprising the steps of:
excising a section of the recipient member to establish the void after the suture path is created; and
removing the component from a donor member after the suture path in the component is created.

13. A method as recited in claim 12 wherein the recipient member and the donor member comprise corneal tissue, and further wherein the creating and excising steps are performed by photoablating corneal tissue.

14. A method as recited in claim 13 wherein the donor member and the recipient member each have a posterior surface and an anterior surface; and wherein, during the creating and excising steps, photoablation is performed in a direction from the posterior surface to the anterior surface.

15. A device for replacing a section of a recipient member with a component from a donor member comprising:
means for defining the section in the recipient member, with said section having a boundary with predetermined dimensions;
means for specifying the component in the donor member, with said component having a boundary with predetermined dimensions substantially compatible with the predetermined dimensions of the section;
means for identifying a suture path in the recipient member and in the component; and
means for creating the suture path in the recipient member and in the component.

16. A device as recited in claim 15 wherein the suture path intersects the respective boundaries.

17. A device as recited in claim 16 wherein the donor member and the recipient member each have a posterior surface and an anterior surface, and wherein the suture path in the recipient member and in the component extends from the respective boundary to the respective anterior surface.

18. A device as recited in claim 16 further comprising means for aligning the suture path in the component with the suture path in the recipient member.

19. A device as recited in claim 15 further comprising:
means for removing the component from the donor member after the suture path is created; and
means for excising the section from the recipient member after the suture path is created.

20. A device as recited in claim 19 wherein the recipient member and the donor member comprise corneal tissue, and further wherein the means for removing, excising and creating include photoablation means.
